(19) United States
(12) Patent Application Publication
(10) Pub. No.: US 2025/0262321 A1
(43) Pub. Date: Aug. 21, 2025

(54) DUAL VECTOR SELF-INACTIVATING CRISPR/CAS9 SYSTEM

(71) Applicants: Chenjian LI, Irvine, CA (US); Sushuang ZHENG, Los Angeles, CA (US)

(72) Inventors: Chenjian LI, Irvine, CA (US); Sushuang ZHENG, Los Angeles, CA (US)

(21) Appl. No.: 18/583,277

(22) Filed: Feb. 21, 2024

(51) Int. Cl. A61K 48/00; A61P 25/28; C12N 9/22; C12N 15/11; C12N 15/86

(52) U.S. Cl. CPC A61K 48/005; A61K 48/0083; A61P 25/28; C12N 9/22; C12N 15/111; C12N 15/86; C12N 2310/20; C12N 2750/14143; C12N 2750/14145

(57) ABSTRACT

A self-inactivating CRISPR/Cas9 delivery system utilizes a dual vector system. The first viral vector includes a unit for expression of a Cas9 nuclease and a nucleotide sequence encoding an sgRNA targeting a specific genomic locus. The second viral vector includes a nucleotide sequence encoding the sgRNA targeting expression of the Cas9 nuclease by the expression unit. The self-inactivating CRISPR/Cas9 dual vector delivery system can be used treating a genetic disease or genetic disorder and for treating a gene-associated disease or condition.

### SEQUENCE LISTING

The instant application contains a Sequence Listing which has been submitted electronically in XML format and is hereby incorporated by reference in its entirety. Said XML copy, created on Feb. 7, 2024, is named DTSN-0001_SL.xml and is 13,792 bytes in size.

The present invention relates to a gene-editing method and viral vectors for use therein. In particular, the invention relates to a method of subjecting a patent to gene-editing using a self-inactivating CRISPR/Cas9 system.

In general, the CRISPR/Cas9 system used in the method according to the invention is a dual vector system that includes a first vector component that comprises a single guide RNA (sgRNA) that targets a gene and provides Cas9 expression, and a second vector component that comprises single guide RNA that targets Cas9. In one embodiment, the dual vector system involves use of a first viral vector for delivering Cas9 (e.g., SaCas9 from Staphylococcus aureus) and an sgRNA targeting HTT gene to provide HTT editing, and a second viral vector for delivering sgRNA against Cas9 to inactivate expression of Cas9.

### BACKGROUND

Genome editing or gene-editing is a powerful technique presenting great possibilities in the treatment of numerous genetic diseases and disorders, as well as many other gene-associated conditions. In particular, the CRISPR/Cas gene-editing system provides great hope for development of treatments for such diseases/disorders/conditions.

In general, the CRISPR/Cas system utilizes two main components: a guide RNA (gRNA) and an associated Cas nuclease (CRISPR associated nuclease), e.g., Cas9 nuclease. The gRNA has two parts, crRNA, which is nucleotide sequence complementary to the target DNA, and tracrRNA, which is a nucleotide sequence that functions as a binding scaffold for the Cas nuclease. A single guide RNA (sgRNA) is a single RNA molecule in which crRNA sequence and tracrRNA sequence are fused together.

During gene-editing, the gRNA/Cas complex recognizes a protospacer adjacent motif (PAM) adjacent the target sequence and the gRNA binds to the target sequence. A double strand break (DSB) at the target sequence is then formed by the Cas9 nuclease. The DSB triggers endogenous DNA repair mechanisms, namely homology directed repair (HDR), and non-homologous end joining (NHEJ). Using this cellular repair machinery, the CRISPR/Cas system can make insertions, replacements, and/or deletions in the DNA sequence. CRISPR/Cas technology and its use as a gene-editing tool in treating genetic disorders is described in detail in, for example, Zhang et al. (WO 2014/093701) and Zhang et al. (WO 2016/020399), hereby incorporated by reference.

As used herein, a genetic disease or genetic disorder is disease resulting from abnormality in a genome. The abnormality can be inherited or acquired. The genetic diseases/disorders can be caused by a mutation in single gene (monogenic disorders), or can be caused by a combination of factors such as mutations in multiple genes or a combination gene mutations and environmental factors (complex or multifactorial disorders), or can be caused by damage to chromosomes. As used herein, a gene-associated disease or condition is a disease that is impacted by the expression of one or more certain genes wherein alteration of the expression of the gene(s) can alleviate the symptoms of the disease, for example, signaling biochemical pathway-associated genes.

Examples of monogenic genetic disorders include cystic fibrosis, myotonic dystrophy, spinal muscular dystrophy, alpha-thalassemia, beta-thalassemia, sickle cell anemia, chronic granulomatous disease Canavan disease, Fanconi anemia, Marfan syndrome, retinitis pigmentosa, fragile X syndrome, Gaucher disease, Huntington's disease, hemochromatosis, congenital deafness, Duchenne muscular dystrophy, familial hypercholesterolemia, Farber disease, Neurofibromatosis type 1, Neurofibromatosis t Becker muscular dystrophy type II, hemophilia A, hemophilia B, Tay-Sachs disease, Wiskott-Aldrich syndrome, Hurler syndrome, Hunter syndrome, Purine nucleoside phosphorylase deficiency, Fabry disease, Pompe disease, Gyrate atrophy, Krabbe disease, Wolman disease, Amyotrophic lateral sclerosis, Spinocerebellar Ataxia, and Sanfilippo disease.

Examples of complex or multifactorial disorders include birth defects, breast cancer, prostate cancer, skin cancer, high blood pressure, high cholesterol, diabetes, Alzheimer disease, schizophrenia, Parkinson's disease, Frontotemporal dementia, and bipolar disorder.

Numerous genetic diseases/disorders and disease-associated genes including signaling biochemical pathway-associated genes are listed in Tables A, B, and C of U.S. Pat. No. 11,708,588, hereby incorporated by reference.

One genetic disease for which CRISPR/Cas technology could lead to beneficial and effective treatment is Huntington's disease (HD). See, e.g., WO 2016/020399. HD is an inherited neurodegenerative disorder, characterized by loss of striatal neurons, which causes the progressive breakdown of nerve cells in the brain resulting in movement disorders. These movement disorders include both involuntary and voluntary movements, such as jerking movements, rigidity, muscle contracture, abnormal eye movements, impaired walking, posture and/or balance, and difficulty with speech or swallowing. Huntington's disease can further cause cognitive disorders such as difficulty organizing/focusing on tasks, perseveration, and lack of impulse control.

In 1993, the Huntingtin gene (HTT) was isolated. Located on chromosome 4, the Huntingtin locus spans 180 kb and consists of 67 exons. The gene codes for production of a protein called “huntingtin”, which is believed to play an important role in nerve cell survival and function. HD is caused by an abnormal version of HTT which exhibits an unstable trinucleotide repeat in the huntingtin gene, i.e., the CAG trinucleotide repeat. The CAG repeat translates as a polyglutamine repeat in the protein product. In normal HTT gene, the range CAG trinucleotide repeats can be fairly broad, for example, 10-26. In the mutant (mHTT) or abnormal HTT gene, the CAG repeats can be >36. HD is autosomal dominant. Thus, an individual need receive only one copy of the abnormal HTT responsible for the excessive CAG repeats from either parent to have Huntington's disease.

As discussed above, CRISPR/Cas technology offers the possibility for the development of treatments for genetic diseases/disorders like HD and other gene-associated conditions. However, a difficulty associated with using CRISPR/Cas technology in gene-editing is long term expression of the CRISPR/Cas, specifically expression Cas nuclease protein. This can lead to undesirable immune responses and off-target cleavages inducing mutagenesis of DNA sequences. Consequently, various techniques have been developed to render Cas expression transient and thereby minimize off-target effects.

Chen, et al., Mol. Ther 24, 1508-1510 (2016), disclose an in vitro study on a self-restricted CRISPR system using a single lentiviral vector format. The study used a modified lentiCRISPR v2 plasmid that contained a guide RNA targeting the P53 gene, and a second guide RNA to target the Cas9 gene. A human hepatoma cell line, Huh7, was transfected with the lentiviral vector. They observed that cells expressing the self-restricted vector exhibited efficient knockout of the P53 gene while reducing the expression levels of SpCas9 protein. Cas9 protein expression was reduced 2 days post-infection. Compared to cells without SpCas9 sgRNA, this system significantly decreased off-target site occurrences.

Petris, et al., Nat. Commun. 8, 15334. (2017), describe the results of study on a self-limiting Cas9 deliver system. The study involved a single lentiviral vector containing Streptocovvus pyogenes Cas9 (SpCas9), a self-limiting sgRNA targeting Cas9, and a second sgRNA targeting a chosen genomic locus, i.e., enhanced green fluorescent protein (EGFP). In 293T cells, they observed that the self-inactivating system was effective in knocking out the endogenous gene and reduced the off-target events. See also, Ceresto et al. (US 2020/0080090), “Self-Limiting Cas9 circuitry for Enhanced Safety (SLiCES) Plasmid and Lentiviral System Thereof.”

Li, et al., Molecular therapy: Methods & clinical development, 12, 111-122 (2018), describe a self-deleting AAV-CRISPR system. A dual-vector system is used wherein one vector expresses SaCas9 driven by a liver-specific promoter and sgRNA for the target gene, and the other vector expresses sgRNA for SaCas9. The AAV-packaged vectors were delivered to liver cells through intraperitoneal injection. The results confirmed that the self-inactivating system effectively knocked out harmful genes associated with the liver and reduced SaCas9 expression to a minimum.

Shen, et al., Nucleic Acids Res 47, e13 (2019), describe a synthetic switch for minimizing CRISPR off-target effects resulting from Cas9 expression by self-restricting both Cas9 transcription, and Cas9 translation. Cas9 sgRNA was used to control transcription of SpCas9 and K-turn/L7Ae was used to regulate translational expression of Cas9. In the design, one vector carried SpCas9 and K-turn/L7Ae elements and another vector carried target gene sgRNA and SpCas9 sgRNA. Despite such strict control of Cas9 expression in the system, they still achieved efficient knockout of the endogenous gene and reduced off-target rates in HEK293T cells.

Kelkar, et al., Molecular therapy 28, 29-41. (2020), describe a single guide RNA system that uses doxycycline (Dox) to deactivate Streptocovvus pyogenes Cas9 (SpCas9) nuclease in a doxycycline-dependent manner. The lentiviral system involved two vectors: (a) a first vector carrying Cas9, a reporter, and an sgRNA targeting the gene of interest, and (b) a second vector carrying a Tet-repressor, Cerulean reporter, and tetracycline/Dox-inducible sgRNA targeting Cas9. The lentiviral system was applied in editing endogenous genes in HEK293T cells.

Merienne, et al., Cell Rep 20, 2980-2991 (2017), describe a self-inactivating Cas9 system (KamiCas9 system) for editing CNS disease genes such as Huntington's disease. A dual-vector system was used, with one vector expressing SpCas9 and the other expressing sgRNA for HTT and sgRNA for Cas9. These vectors were transfected into HEK293T cells carrying 82Q and primary striatal neurons from HD mice KI140Q. Results showed that this system effectively knocked out the HTT gene and significantly reduced expression levels of Cas9.

Li, et al., Human gene therapy, 30(11) (2019), describe self-destructing CRISPR/Cas constructs for targeted gene editing in the retina. The system was a dual AAV vector system with a first vector delivering Streptocovvus pyogenes Cas9 (SpCas9) and a second vector delivering sgRNAs against SpCas9 and a target locus (yellow fluorescent protein (YFP)). The system achieved efficient editing in YFP with a decreased Cas9 expression.

An objective of the present invention is to provide a self-inactivating CRISPR/Cas9 system that minimizes off-target effects and provides better control of the gene-editing process.

### BRIEF DESCRIPTION OF THE DRAWINGS

FIG. 1 shows schematic representations of vector 1 and vector 2 exemplary embodiments according to the invention. The embodiment of vector 1 includes CMV (cytomegalovirus)-driven SaCas9 and U6-driven sgRNA targeting HTT gene between two ITRs (inverted terminal repeat). In vector 1 “NLS” refers to nuclear localization signal sequence and “HA” refers to the glycol protein human influenza hemagglutinin. The first embodiment of vector 2 includes CMV (cytomegalovirus)-driven EGFP (reporter gene) and U6-driven sgRNA targeting Cas9 between two ITRs (inverted terminal repeat). The second embodiment of vector 2 includes CMV (cytomegalovirus)-driven mCherry (reporter gene) and U6-driven sgRNA targeting Cas9 between two ITRs. The third embodiment of vector 2 includes only U6-driven sgRNA targeting Cas9 between two ITRs without any reporter gene.

FIG. 2 shows results of Western blot tests for SaCas9 and HTT using SaCas9-gRNAs (sgRNA1 to sgRNA10) according to the invention in HEK293T cells.

FIGS. 3A and 3B illustrate results of HD mice (mouse model BAC226Q) injected with AAV9-SaCas9-HTTg1/gCas9 or AAV9-SaCas9-HTTg1 (control group: AAV9-SaCas9). FIG. 3A: fluorescent micrograph of brain samples (scale bar: 50 μm). FIG. 3B: graphical representation of quantitative analysis of fluorescence intensity of FIG. 3A and mHTT aggregation signals.

FIGS. 4A and 4B presents graphical representations of sequential timepoints Motor phenotype analysis of 1st-month-old virus injected BAC226Q mice.

### DETAILED DESCRIPTION OF THE INVENTION

The invention is a self-inactivating CRISPR/Cas9 system that utilizes a dual vector system (e.g., a dual AAV vector system). The system includes a single guide RNA (sgRNA) targeting a specific genomic locus (for example, the HTT gene (HTTg1)) and delivering Cas9 (e.g., SaCas9) and another sgRNA targeting Cas9 (e.g., SaCas9), i.e., guide Cas9 or gCas9. The system components are packaged and implemented through dual vectors. A first vector delivers Cas9 and sgRNA against the target gene and the second vector delivers sgRNA against Cas9.

Upon deployment of the dual AAV vector system, the sgRNA targeting the specific genomic locus (e.g., HTTg1) binds to the target gene and Cas9 begins to express, leading to destruction of the target gene. Excessive Cas9 expression is then disrupted simultaneously by the sgRNA targeting Cas9 (e.g., SaCas9), i.e., gCas9. The gCas9 targets the N terminus of Cas9 RuvC-I domain which plays crucial role in nuclease activity, thereby disrupting expression of Cas9 and achieving transient expression of Cas9. As a result, this self-inactivating CRISPR/Cas9 system attenuates Cas9 expression time, resulting in reduction or complete elimination of off-target events.

The self-inactivating CRISPR/Cas9 system according to the invention offers advantages in comparison to other attempts to provide for self-inactivation. For example, in comparison to a system wherein two sgRNAs are delivered by one vector and Cas9 alone is delivered by another vector, the self-inactivating CRISPR/Cas9 system according to the invention provides more control over both efficient editing of target gene and expression control of Cas9 nuclease by adjusting the molar ratio between the two vectors. Further, in systems where the gRNA for the target gene and the gRNA for Cas9 are packaged together with Cas9 in a single vector, there is a disadvantage Cas9 itself can face the risk of being knocked out during plasmid passaging and viral packaging. As a result, other molecules may be used to suppress Cas9 expression, such as using TetR to suppress expression of Cas9 driven by CMV-TetO or anti-CRISPR molecules to block self-targeting. However, such approaches may introduce unnecessary molecules or cause unnecessary complications.

According to a first aspect, the invention is directed to a self-inactivating CRISPR/Cas9 delivery system comprising:
a first viral vector having an expression unit for expression of a Cas9 nuclease and a nucleotide sequence encoding an sgRNA targeting a specific genomic locus; and
a second viral vector having a nucleotide sequence encoding the sgRNA targeting expression of the Cas9 nuclease by the expression unit.

According to a further aspect, the invention is directed to a self-inactivating CRISPR/Cas9 delivery system comprising:
a first viral vector having an expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said first viral vector further comprises a CMV promoter to drive expression the Cas9 nuclease.

According to a further aspect, the invention is directed to a self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said second viral vector does not include a nucleotide sequence encoding a reporter gene.

According to a further aspect, the invention is directed to a self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said first and second viral vectors are not AAV8 viral vectors, and preferably are AAV9, AAV PHP.B, or AAV PHP.eB viral vectors, particularly AAV9 viral vectors.

According to a further aspect, the invention is directed to a self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein the sgRNA targeting expression of the Cas9 nuclease by the expression unit comprises a nucleotide sequence selected from SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, or a nucleotide sequence having at least 95% homology, at least 96% homology, at least 97% homology, at least 98% homology, or at least 99% homology with any of SEQ ID NO:3 to SEQ ID NO.12, and
the specific genomic locus is selected from neurodegenerative disease related to genes in the CNS, wherein preferably the neurodegenerative disease is selected from Huntington's disease, Alzheimer's disease, Parkinson's disease, Amyotrophic Lateral Sclerosis (ALS), Frontotemporal dementia (FTD) (aka Pick's disease), spinocerebellar ataxi (SCA), e.g., types 1 to 17, Retinitis Pigmentosa, or Age-related macular degeneration (AMD), particularly Huntington's disease.

According to a further aspect, the invention is directed to any of the self-inactivating CRISPR/Cas9 delivery system described above, wherein the Cas9 nuclease is SaCas9.

According to a further aspect, the invention is directed to any of the self-inactivating CRISPR/Cas9 delivery systems described above, wherein the two vectors are both AAV vectors, e.g. AAV9 vectors.

According to a further aspect, the invention is directed to any of the self-inactivating CRISPR/Cas9 delivery systems described above, wherein the specific genomic locus is selected from neurodegenerative disease related to genes in the CNS.

According to a further aspect, the invention is directed to any of the self-inactivating CRISPR/Cas9 delivery systems described above wherein the sgRNA targeting a specific genomic locus targets a gene associated with Huntington's disease, Alzheimer's disease, Parkinson's disease, Amyotrophic Lateral Sclerosis (ALS), Frontotemporal dementia (FTD) (aka Pick's disease), spinocerebellar ataxi (SCA), e.g., types 1 to 17, Retinitis Pigmentosa, or Age-related macular degeneration (AMD).

According to a further aspect, the invention is directed to any of the self-inactivating CRISPR/Cas9 delivery systems as described above, wherein the sgRNA targeting a specific genomic locus targets Huntingtin (HTT) gene.

According to a further aspect, the invention is directed to any of the self-inactivating CRISPR/Cas9 delivery systems as described above, wherein nucleotide sequence encoding the sgRNA targeting a specific genomic locus comprises a sequence according to SEQ ID NO.1 or SEQ ID NO.2, or a nucleotide sequence having at least 95% homology, at least 96% homology, at least 97% homology, at least 98% homology, or at least 99% homology with SEQ ID NO.1 or SEQ ID NO.2.

According to a further aspect, the invention is directed to any of the self-inactivating CRISPR/Cas9 delivery systems as described above, wherein nucleotide sequence encoding the sgRNA targeting a specific genomic locus comprises a sequence having the same function as the nucleotide sequence of SEQ ID NO:1 or SEQ ID NO:2, or a nucleotide sequence obtained from the nucleotide sequence of SEQ ID NO:1 or SEQ ID NO:2 by deletion, substitution or addition of 1, 2, 3, 4, 5 or 6 bases, and having the same function as the nucleotide sequence of SEQ ID NO:1 or SEQ ID NO:2.

According to another aspect, the invention is directed to any of the self-inactivating CRISPR/Cas9 delivery systems as described above, wherein the nucleotide sequence encoding the sgRNA targeting expression of the Cas9 nuclease is a sequence selected from SEQ ID NO.3, SEQ ID NO.4, SEQ ID NO.5, SEQ ID NO.6, SEQ ID NO.7, SEQ ID NO.8, SEQ ID NO.9, SEQ ID NO.10, SEQ ID NO.11, and SEQ ID NO.12, or a nucleotide sequence having at least 95% homology, at least 96% homology, at least 97% homology, at least 98% homology, or at least 99% homology with any of SEQ ID NO:3 to SEQ ID NO.12.

According to another aspect, the invention is directed to any of the self-inactivating CRISPR/Cas9 delivery systems as described above, wherein the nucleotide sequence encoding the sgRNA targeting expression of the Cas9 nuclease is a comprises a sequence having the same function as the nucleotide sequence of SEQ ID NO:3 to SEQ ID NO:12, or a nucleotide sequence obtained from the nucleotide sequence of SEQ ID NO:3 to SEQ ID NO:12 by deletion, substitution or addition of 1, 2, 3, 4, 5 or 6 bases, and having the same function as the nucleotide sequence of SEQ ID NO:3 to SEQ ID NO:12.

According to another aspect, the invention is directed to a pharmaceutical composition comprising a self-inactivating CRISPR/Cas9 delivery system according to any of the self-inactivating CRISPR/Cas9 delivery systems as described above and a pharmaceutical acceptable carrier or excipient.

According to another aspect, the invention is directed to a method for treating a genetic disease or genetic disorder comprising administering to a patient in need thereof any of the self-inactivating CRISPR/Cas9 delivery systems as described above.

According to another aspect, the invention is directed to a method for treating, a gene-associated disease or condition comprising administering to a patient in need thereof any of the self-inactivating CRISPR/Cas9 delivery systems as described above.

According to another aspect, the invention is directed to a method for treating a Huntington's disease comprising administering to a patient in need thereof any of the self-inactivating CRISPR/Cas9 delivery systems as described above.

In accordance with the invention, the Cas9 nuclease can be, for example, Cas9 from Staphylococcus aureus (SaCas9), from Streptococcus pyogenes (SpCas9), Cas9 from Streptococcus thermophilus (StCas9), Cas9 from Campylobacter jejuni (CjCas9), and Cas9 from Neisseria meningitidis (NmCas9). It is also possible that other Cas nucleases can be used, such as Cas12a, Cas12b, Cas12e, Cas12j, Cas12f1, Cas13a, and Cas14a. In each case, the sgRNA targeting the Cas nuclease must be able to effectively target and deactivate the particular Cas nuclease involved.

Species that produce naturally occurring Cas9 are described in Chylinski et al., RNA Biology 2013, 10:5, 727-737.

In accordance with the invention, the vector containing the single guide RNA (sgRNA) targeting HTT gene and delivering SaCas9 is, for example, selected from the sequences presented in the following Table.

| Name | Sequence | SEQ ID NO: |
|---|---|---|
| HTTg1 | 5′-TGGAAAAGCTGATGAAGGCCT-3′ | SEQ ID NO: 1 |
| HTTg2 | 5′-GAAGGCCTTCATCAGCTTTTC-3′ | SEQ ID NO: 2 |

The sgRNA targeting HTT and delivering SaCas9 can further be a sequence having at least 95%, at least preferably 97%, very preferably at least 98%, and especially at least 99% homology with either of SEQ ID NO:1 or SEQ ID NO:2. The sgRNA targeting HTT and delivering SaCas9 can further be a sequence comprising a sequence having the same function as the nucleotide sequence of SEQ ID NO:1 or SEQ ID NO:2, or a nucleotide sequence obtained from the nucleotide sequence of SEQ ID NO:1 or SEQ ID NO:2 by deletion, substitution or addition of 1, 2, 3, 4, 5 or 6 bases, and having the same function as the nucleotide sequence of SEQ ID NO:1 or SEQ ID NO:2.

In accordance with the invention, the vector targeting Cas9, particularly SaCas9, employs a sgRNA nucleotide sequence for Cas9, particularly SaCas9, which is, for example, selected from the sequences presented in the following Table.

| Name | Sequence | SEQ ID NO: |
|---|---|---|
| gRNA1 | 5′-GGTGATGCCGATGTCCAGGCC | SEQ ID NO: 3 |
| gRNA2 | 5′-GTGAGCCAGAAGCTGAGCGAGG | SEQ ID NO: 4 |
| gRNA3 | 5′-GAGGTCCCTCATAGTAGGTCCG | SEQ ID NO: 5 |
| gRNA4 | 5′-GCTCAGGATGAAGTCGTCCAC | SEQ ID NO: 6 |
| gRNA5 | 5′-GTTGTTCAGCAGATCTTCCAG | SEQ ID NO: 7 |
| gRNA6 | 5′-GACTTCATCAACCGGAACCTG | SEQ ID NO: 8 |
| gRNA7 | 5′-GTGCCCGAGATCGAAACCGAGC | SEQ ID NO: 9 |
| gRNA8 | 5′-GATGGAACAGTACGGCGACGAG | SEQ ID NO: 10 |
| gRNA9 | 5′-GACGCCATTGTCCAGGTACACG | SEQ ID NO: 11 |
| gRNA10 | 5′-GAACATGATCGACATCACCTACC | SEQ ID NO: 12 |

These sequences are described Li, et al., Molecular therapy: Methods & clinical development, 12, 111-122 (2018), describe a self-deleting AAV-CRISPR system.

The sgRNA nucleotide sequence targeting SaCas9 can further be a sequence having 95%, preferably 97%, especially 98% homology with any of SEQ ID NO:3 to SEQ ID NO:12. The sgRNA targeting SaCas9 can further be a sequence comprising a sequence having the same function as the nucleotide sequence of SEQ ID NO:3 to SEQ ID NO:12, or a nucleotide sequence obtained from the nucleotide sequence of SEQ ID NO:3 to SEQ ID NO:12 by deletion, substitution or addition of 1, 2, 3, 4, 5 or 6 bases, and having the same function as the nucleotide sequence of SEQ ID NO:3 to SEQ ID NO:12.

The viral vectors for use with the invention can be selected from AAV, lentiviral vectors, adenovirus vectors, herpesvirus vectors, poxvirus vectors, baculovirus vectors, and papillomavirus vectors.

The viral vectors for use in the invention are preferably adeno-associated (AAV) vectors, particularly AAV vectors of human origin. In particular, the AAV vector is selected from the group comprising serotypes AAV1, AA2, AAV3, AAV4, AAV5, AAV6, AAV7, AAV8, AAV9, AAV PHP.B, and AAV PHP.eB. In a preferred situation, the AAV vector is not AAV8. In a further preferred embodiment, the AAV vector is AAV9, AAV PHP.B, or AAV PHP.eB, especially AAV9.

The invention further includes pharmaceutical compositions. A pharmaceutical composition according to the invention comprises the self-inactivating CRISPR/Cas9 delivery system in the form of dual viral vectors, a pharmaceutically acceptable carrier, and optionally pharmaceutically acceptable diluents, or excipients known to those skilled in the art.

Examples of pharmaceutically acceptable carriers include aqueous carries (e.g., water or salt solutions such as phosphate-buffered saline), alcohols, and polyethylene glycols. Pharmaceutically acceptable diluents/excipients can include, for example, wetting agents, emulsifying, and suspending agents, salt for influencing osmotic pressure, and/or buffers. For example, the diluent/excipient can be selected from one or more of polyethylene glycol, propylene glycol, vegetable oil, and mineral oil. In a specific example, the preservative is selected from one or more of sorbic acid, methyl sorbate, methyl paraben, ethyl paraben, propyl paraben, butyl paraben, benzyl paraben, sodium methylparaben, benzoic acid, and benzyl alcohol. In another one of the specific examples, the buffering agent is selected from one or more of sodium hydrogen phosphate, sodium dihydrogen phosphate, sodium citrate, sodium tartrate, and sodium acetate. In another one of the specific examples, the disintegrant is selected from one or more of cross-linked sodium carboxymethyl cellulose, sodium carboxymethyl starch, cross-linked polyvinylpyrrolidone, or low-substituted hydroxypropyl cellulose. In another one of the specific examples, the antioxidant is selected from one or more of ethylenediaminetetraacetic acid, ethylenediaminetetraacetic acid disodium salt, dibutylhydroxytoluene, glycine, inositol, ascorbic acid, sodium ascorbate, lecithin, malic acid, hydroquinone, citric acid, succinic acid, and sodium metabisulfite. In another one of the specific examples, the co-suspension agent is selected from one or more of beeswax, ethyl hydroxyethyl cellulose, chitin, chitosan, methyl cellulose, carboxymethyl cellulose, agar, hydroxypropyl methyl cellulose, and xanthan gum. In another one of the specific examples, the colorant is selected from one or more of carbon black, iron black, iron brown, iron red, and titanium dioxide. In another one of the specific examples, the excipient is selected from one or more of mannitol, glucose, lactose, dextran, dextrose, and sodium chloride.

The invention further includes methods of treatment. Thus, the invention includes a method for treating a genetic disorder/disease comprising administering to a patient in need thereof an effective amount of any of the self-inactivating CRISPR/Cas9 delivery systems as described above.

According to one method embodiment, the invention includes a method for treating a genetic disorder/disease neurodegenerative disease related to genes in the CNS comprising administering to a patient in need thereof an effective amount any of the self-inactivating CRISPR/Cas9 delivery systems as described above. According to another method embodiment, the invention includes a method for treating neurodegenerative disease is selected from Huntington's disease, Alzheimer's disease, Parkinson's disease, Amyotrophic Lateral Sclerosis (ALS), Frontotemporal dementia (FTD) (aka Pick's disease), spinocerebellar ataxi (SCA), particularly types 1 to 17, Retinitis Pigmentosa, and Age-related macular degeneration (AMD) comprising administering to a patient in need thereof an effective amount any of the self-inactivating CRISPR/Cas9 delivery systems as described above.

In particular, the invention includes a method for treating a Huntington's disease comprising administering to a patient in need thereof an effective amount any of the self-inactivating CRISPR/Cas9 delivery systems as described above.

The term “an effective amount” refers to an amount necessary to obtain a beneficial or desired physiological effect. The physiological effect may be achieved by one application dose or by repeated applications (e.g., daily over several days, weekly, monthly). The administered dosage will, of course, vary depending upon various factors based on the particular composition administered, the mode of administration, the disease/disorder to be treated, and the patient (e.g., human) to be treated, and can be adjusted by one skilled in the art. Such factors may include, for example, the composition's physiological characteristics; age, health, sex, and weight of the subject; nature/extent of the symptoms; any concurrent treatment; frequency of administration; and desired effect.

Suitable dose ranges for are generally about 10⁹ to 10¹⁴ (e.g., 10⁹, 10¹⁰, 10¹¹, 10¹², 10¹³, or 10¹⁴) viral genomes or infectious units of viral vector per dose, preferably 10⁹ to 10¹³ viral genomes or infectious units of viral vector per dose, more preferably 10¹⁰ to 10¹² viral genomes or infectious units of viral vector per dose. It should be understood that the aforementioned dosage ranges are merely exemplary dosage. Those skilled in the art will understand that this dosage may be varied in light of the factors discussed above.

The mode of administration used in the method of treatment include all known administration modes used for viral vector systems. In the case of a method for treatment of Huntington's disease, the self-inactivating CRISPR/Cas9 delivery systems can, for example, by administered by local administration to deliver the vector system to the striatum and/or cortical regions of the brain, e.g., injection in the human striatum. Brain stereotaxic injection to striatum and cortex is preferred and the delivery method can be, for example, without limitation, intraperitoneal injection, subarachnoid injection, lateral ventricular injection, cerebellar medullary pool injection intravenous injection, or another acceptable delivery method.

The above description will be more fully understand when considered in conjunction with the following Examples and accompanying figures. The following Examples are exemplary in nature with regards to methods for practicing the invention are not intended to be limiting.

### EXAMPLES

The following examples are intended to exemplify the invention and are not to be as construed as limiting the invention.

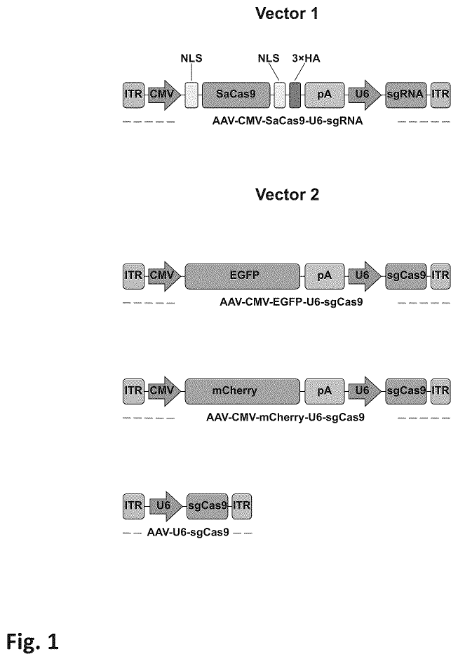

### Vector Cloning (See FIG. 1)

A single vector AAV-SaCas9 system containing Cas9 from Staphylococcus aureus (SaCas9) and its sgRNA scaffold was obtained from Addgene (plasmid #61591). sgRNAs targeting human HTT and SaCas9 were designed based on PAM sequence (5′-NNGRRT-3′) and by CRISPR RGEN Tools (http://www.rgenome.net/). AAV-EGFP-sgCas9 or AAV-mCherry-sgCas9 which contain EGFP or mCherry were constructed by replacing SaCas9 on vector AAV-SaCas9. Top and bottom strands of oligos for each sgRNA were phosphorylated and annealed into duplex. sgRNA for HTT was inserted into AAV-SaCas9 vector through BsaI enzyme digestion. Cas9 was replaced by EGFP or mCherry through AgeI and EcoRI enzyme digestion to obtain vector AAV-EGFP and AAV-mCherry, respectively. sgCas9 was also inserted into AAV-GFP and AAV-mCherry vector through BsaI enzyme digestion. Transformation of ligation product into Stbl3™ competent cells and isolated the plasmid DNA from cultures by using a QIAprep spin miniprep kit (QIAGEN, 27104) according to the manufacturer's instructions. Sequences of sgRNAs were verified by sequencing from the LKO.1 5′ primer: 5′-GAC TAT CAT ATG CTT ACC GT-3′ (SEQ ID NO:13). Sequences of EGFP were verified using EGFP-forward primer: 5′-CGA AGG CTA CGT CCA GGA GC-3′ (SEQ ID NO:14). Sequences of mCherry were verified using mCherry-forward primer: 5′-ACA ACC GGT ATG GTG AGC AAG-3′ (SEQ ID NO:15).

FIG. 1 shows schematic representations of vector 1 and vector 2 exemplary embodiments according to the invention. The embodiment of vector 1 includes CMV (cytomegalovirus)-driven SaCas9 and U6-driven sgRNA targeting HTT gene between two ITRs (inverted terminal repeat). The first embodiment of vector 2 includes CMV (cytomegalovirus)-driven EGFP (reporter gene) and U6-driven sgRNA targeting Cas9 between two ITRs (inverted terminal repeat). The second embodiment of vector 2 includes CMV (cytomegalovirus)-driven mCherry (reporter gene) and U6-driven sgRNA targeting Cas9 between two ITRs. The third embodiment of vector 2 includes only U6-driven sgRNA targeting Cas9 between two ITRs without any reporter gene.

While these vectors used the CMV and U6 promotors, one skilled in the art is well aware other suitable promoters for both the first vector and the second vector. For example, CaMKII, Synapsin, EF-1α, NSE, or PGK promoter could be used to drive Cas9 and reporter protein expression, and H1 or 7SK promoter could be used to drive sgRNA expression.

In terms of promoter for the first viral vector that includes the expression unit for expression of a Cas9 nuclease and the nucleotide sequence encoding an sgRNA targeting a specific genomic locus, preferably a CMV promoter is utilized.

### AAV Vector Production and Purification

AAV plasmids were extracted by EndoFree plasmid maxi kit (QIAGEN, 12362) for following virus production and purification. HEK293T cells were plated on 15-cm dishes and transfected at 80% confluence with linear polyethylenimine (PEI, Sigma-Aldrich, 765090). The transfection mixture was prepared with 70 μg AAV9 vector (AAV-SaCas9-HTTg1, AAV-SaCas9, AAV-GFP-sgCas9 or AAV-GFP), 200 μg Ad-Helper plasmid, 70 μg AAV-Rep/Cap plasmid and PEI (1 μg/μL), the PEI to DNA ratio was 5:1 (v/g) in our experiment. The mixture was filled to 50 mL with DMEM and sit room temperature for 15 min. Cells were harvested 60 h after transfection and re-suspended in 5 mL cell lysis buffer (150 mM NaCl, 20 mM Tris, pH 8.0). Freeze-thaw the cell lysate completely for 3 times between lipid nitrogen and 37° C. water bath. Cell lysate was added with MgCl₂ and Benzonase (Sigma, E8263-25k) to a final concentration of 1 mM and 250 U/mL respectively, followed by incubation at 37° C. for 15 min.

The supernatant was collected after centrifugation of the cell lysate at 4000 rpm for 30 min at 4° C. The viral solution was transferred on the top layer of the discontinuous iodixanol gradient solution, which is listed in the order of 6 mL of 17% (5 mL 10×PBS, 0.05 mL 1 M MgCl₂, 0.125 mL 1 M KCl, 10 mL 5 M NaCl, 12.5 mL Optiprep (Sigma, D1556) and H2O up to 50 mL), 6 mL 25% (5 mL 10×PBS, 0.05 mL 1 M MgCl₂, 0.125 mL 1 M KCl, 20 mL Optiprep and H2O up to 50 mL), 5 mL of 40% (5 mL 10×PBS, 0.05 mL 1 M MgCl₂, 0.125 mL 1 M KCl, 33.3 mL Optiprep and H2O up to 50 mL) and 4 mL of 60% (0.05 mL 1 M MgCl₂, 0.125 mL 1 M KCl, 50 mL Optiprep) from the bottom. The gradient was centrifuged at 53000 rpm for 160 min at 14° C. and the viral fraction was harvested in the 40% layer with a syringe.

The viral fraction was transferred into the activated concentrated column Amacon 100K filter (Millipore Sigma, UFC910008) and filled with PBS solution containing F188 (1:10000, Polomaxer, Sigma). Then, it was centrifuged at 3500 rpm for 20 min at 4° C. The filtrate was discarded, and the viral fraction was again filled with F188-containing PBS solution to be repeatedly spun and added PBS for 3 times. The filtrate was discarded again, and the viral fraction was mixed with PBS to be concentrated by centrifugation at 3500 rmp for 20 min at 4° C. until the viral volume was about 500 μL. Virus titer was measured by quantitative PCR. Virus quality was determined by SDS-PAGE followed by Coomassie blue staining which would show virus capsid protein.

A single vector AAV-SaCas9-gHTT system containing Cas9 from Staphylococcus aureus (SaCas9) and its sgRNA scaffold was obtained from Addgene (plasmid #61591). Single guide RNAs targeting human HTT and SaCas9 were designed based on PAM sequence (5′-NNGRRT-3′) and by CRISPR RGEN Tools (http://www.rgenome.net/). Top and bottom strands of oligos for each sgRNA were phosphorylated and annealed into duplex. Single guide RNAs (HTTg1/HTTg2) were inserted into AAV-SaCas9 vector through BsaI enzyme digestion. Sequences of gRNA were verified by sequencing from the LKO.1 5′ primer: 5′-GACTATCATATGCTTACCGT-3′ (SEQ ID NO:13). Regarding AAV vector production and purification, see above.

### Culture and Transfection of HEK293T Cell

HEK293T cells were cultured in Dulbecco's modified Eagle's medium (DMEM, Thermo Fisher) supplemented with 10% (v/v) fetal bovine serum (FBS, Thermo Fisher), 1% (v/v) penicillin-streptomycin (Thermo Fisher) and 1% (v/v) L-glutamine (Thermo Fisher) in a humidified 5% CO₂ atmosphere at 37° C. Cells were co-transfected with AAV-SaCas9-HTTg1/AAV-GFP-gCas9 and pEGFPc3-120 CAG with 0.8 μg of each vector at 6-well plate at 70% confluency with lipofectamine 2000 (Invitrogen, 11668030).

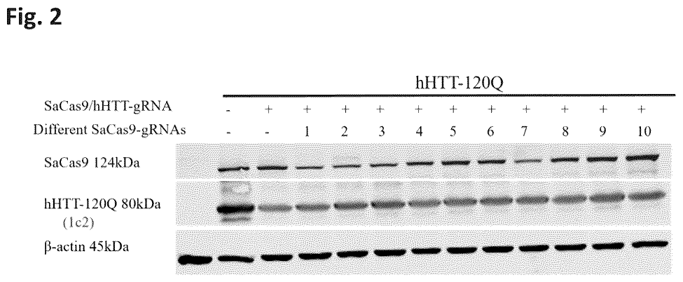

### Western Blot (See FIG. 2).

Lysates from 293T cells were prepared in Triton X-100 buffer (150 mM NaCl, 50 mM Tris-HCl, pH 7.4, 1 mM EDTA, 1% Triton X-100). The lysates were incubated at 4° C. for 30 min, followed by centrifugation at 4° C. for 20 min at 16,100 g to remove insoluble components. Protein concentration was measured by BCA protein assay. Protein samples were prepared for loading by heating in 4×SDS sample buffer and heated for 10 min at 95° C. A total amount of 30 μg of protein was loaded onto SDS-PAGE gels using running buffer, and wet-transferred to an Immobilon-FL PVDF membrane. Blots were incubated with Odyssey blocking buffer (LI-COR, 927-40000) for 60 min. After washing with TBST, blots were incubated with primary antibodies diluted in blocking buffer at 4° C. overnight. Blots were washed 3 times (10 min each) and incubated with fluorescently labeled IRDye 680RD goat anti-mouse (1:10000, LI-COR, 926-68070) or goat anti-rabbit (1:10000, LI-COR, 926-68071) secondary antibodies in TBST for 60 min at room temperature. Protein were detected in the 700 nm channel using Odyssey CLx imager (LI-COR). Following primary antibodies were used: 1C2 (1:5000, mouse, Millipore, MAB1574), HA (1:2000, mouse, Abcam, ab18181), β-actin (1:2000, rabbit, Cell Signaling, 4970). As shown in FIG. 2, Western blot analysis validated that the tested sequences, sgRNA1 to sgRNA10, were able to knock out SaCas9 without compromising the gene editing efficiency of HTT-gRNA to hHTT-120Q.

### Stereotaxic Injection

Stereotaxic administration of AAV vectors were performed on 1-month-old of BAC226Q mice. Mice were anesthetized with 1.5% isoflurane inhalation and stabilized in a stereotaxic instrument (RWD, 68019). Small holes were drilled in the skull at both side of striatum and cortex. A total of 2.5 μL AAV was injected into mouse striatum at a rate of 0.3 μL/min (coordinates: +0.8 mm rostral to Bregma, ±2.1 mm lateral to medial and −3.1 mm ventral from brain surface). And 0.5 μL of AAV was injected into mouse primary motor cortex at 0.1 μL/min (coordinates: +1.5 mm rostral to Bregma, +1.5 mm lateral to medial and −1.0 mm ventral from brain surface). Two viruses were co-injected at 1:1 molar ratio. Each virus titer was 2×10¹² viral genomes/mL with the total volume of 3 μL, therefore 6×10⁹ viral genomes of each virus were injected into one mouse brain. Injection was performed through a microinjection pump (RWD, 788130)-connected Hamilton syringe. A 1701 Hamilton microsyringe (Hamilton, 7853-01) with 33-gauge needle (Hamilton, 7803-05) was used to deliver the virus.

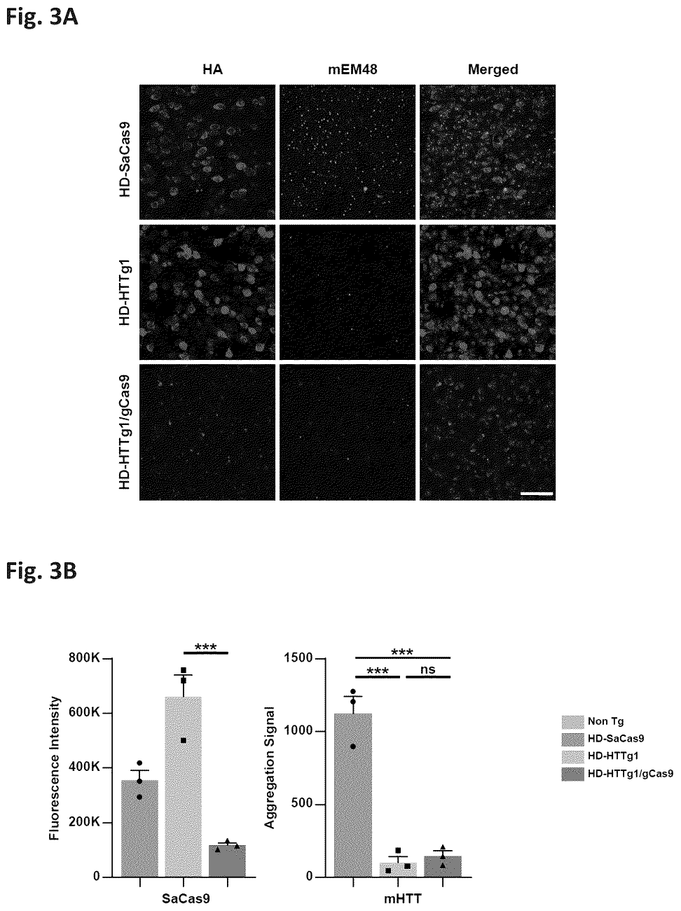

### Immunofluorescent Staining (FIGS. 3A and 3B).

Mice were anesthetized, perfused with fresh 4% paraformaldehyde in PBS, and post-fixed overnight in the same fixative. Fixed brains were sliced at 40 μm thickness with a vibrating blade microtome (Leica, VT1200S). The brain slices were incubated in PBST (0.3% Triton X-100) for 30 min in room temperature and then blocked with 10% goat serum in PBS supplemented with 0.1% Triton X-100 for 60 min at room temperature. Following incubation of brain slices with primary antibodies at 4° C. overnight and washes, fluor-conjugated secondary antibodies (anti-mouse or anti-rabbit Alexa Fluor 594 or 647, 1:1000, Thermo Fisher Scientific) were added to the samples for 60 min at room temperature. Images were taken by Zeiss LSM 710. Following primary antibodies were used: mEM48 (1:1000, mouse, Millipore, MAB5374), HA (1:1000, rabbit, Cell Signaling Technology, 3724). As shown in FIGS. 3A and 3B, BAC226Q mice, after injection of AAV9-SaCas9-HTTg1, showed significant decrease of mHTT. And injection of AAV9-SaCas9-HTTg1/gCas9 showed similar reduction of mHTT along with decreased SaCas9 expression.

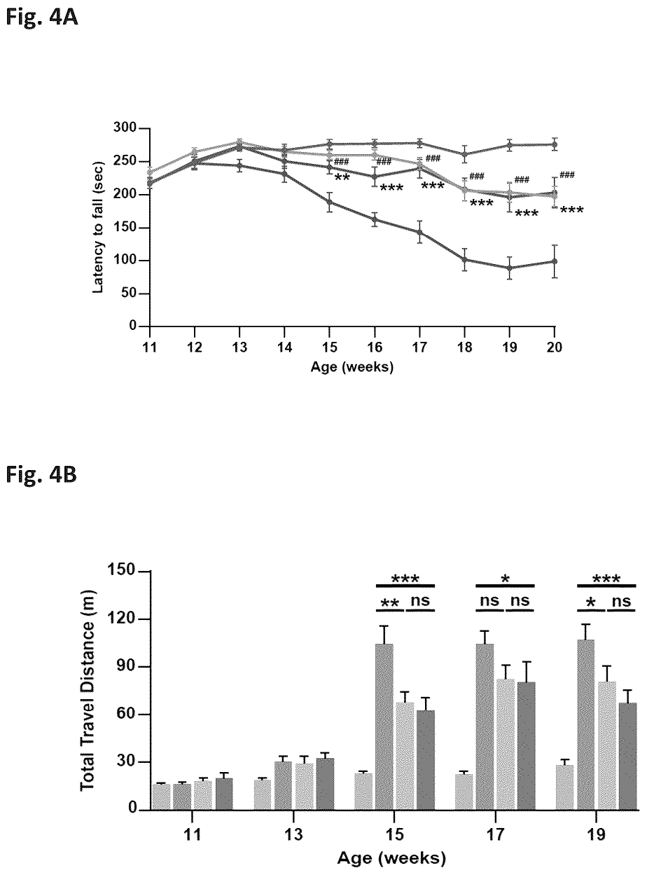

### Rota-Rod Test (FIG. 4A).

Mice were trained on a rota-rod treadmill (Med Associates, Inc., ENV-574M) for 3 trials per day for 3 consecutive days. Mice were trained at a constant speed of 10 rpm/min for 1 min per trial. During the training trials, mice that fell were gently returned to the rota-rod treadmill. Animals were tested for 3 trials per day for 3 consecutive days every week, allowing at least 15 min of rest between each trial. Mice were tested with the rota-rod accelerating from 5 to 40 rpm with a maximum period of 300 sec. The latency to fall was recorded and the mean of all trials was used for statistical analysis. As shown in FIG. 4A, BAC226Q mice, after injection of AAV9-SaCas9-HTTg1 or AAV9-SaCas9-HTTg1/gCas9, showed significant improvement in duration time on rod.

### Open-Field Test (FIG. 4B)

The open field consisted of a clear glass box (28×28 cm, Med Associates, Inc., ENV-510). Mice were tested for 3 trials per day for 3 consecutive days every 2 weeks. Mice were put into the open-field boxes over a 10 min period in every trial according to an established protocol. The total travel distance was recorded and the mean of all trials was used for statistical analysis. As shown in FIG. 4B, both the AAV9-HTTg1/gCas9 and AAV9-HTTg1 injected groups exhibited reduced hyperactivity compared with the HD-SaCas9 control group at relative timepoints.

Although the foregoing subject matter has been described in some detail by way of illustration and example for purposes of clarity of understanding, it will be understood by those skilled in the art that certain changes and modifications can be practiced within the scope of the appended claims.

All publications, patent applications, patents, and other references mentioned herein and incorporated by reference to the same extent as if each individual publication, patent application, patent, and other reference was specifically and individually indicated to be incorporated by reference.

1. A self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said sgRNA targeting expression of the Cas9 nuclease by the expression unit comprises a nucleotide sequence selected from SEQ ID NO.3, SEQ ID NO.4, SEQ ID NO.5, SEQ ID NO.6, SEQ ID NO.7, SEQ ID NO.8, SEQ ID NO.9, SEQ ID NO.10, SEQ ID NO.11, and SEQ ID NO.12, or a nucleotide sequence having at least 95% homology, at least 96% homology, at least 97% homology, at least 98% homology, or at least 99% homology with any of SEQ ID NO:3 to SEQ ID NO.12, and
said specific genomic locus is selected from neurodegenerative disease related to genes in the CNS.

2. The delivery system according to claim 1, wherein said sgRNA targeting expression of the Cas9 nuclease by the expression unit comprises a nucleotide sequence selected from SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

3. The delivery system according to claim 1, wherein said neurodegenerative disease is selected from Huntington's disease, Alzheimer's disease, Parkinson's disease, Amyotrophic Lateral Sclerosis (ALS), Frontotemporal dementia (FTD) (aka Pick's disease), spinocerebellar ataxi (SCA), particularly types 1 to 17, Retinitis Pigmentosa, and Age-related macular degeneration (AMD).

4. The delivery system according to claim 3, wherein said neurodegenerative disease is Huntington's disease.

5. The self-inactivating CRISPR/Cas9 delivery system according to claim 1, wherein said first and second viral vectors are selected from AAV, lentiviral vectors, adenovirus vectors, herpesvirus vectors, poxvirus vectors, baculovirus vectors, and papillomavirus vectors.

6. The delivery system according to claim 5, wherein first and second viral vectors are selected from AAV vectors.

7. The delivery system according to claim 6, wherein said first and second viral vectors are selected from AAV1, AA2, AAV3, AAV4, AAV5, AAV6, AAV7, AAV8, AAV9, AAV PHP.B and AAV PHP.eB.

8. The delivery system according to claim 6, wherein said first and second viral vectors are not AAV8 viral vectors.

9. The delivery system according to claim 1, wherein said first and second viral vectors are AAV9, AAV PHP.B, or AAV PHP.eB viral vectors.

10. The delivery system according to claim 9, wherein said first and second viral vectors are AAV9 viral vectors.

11. The delivery system according to claim 1, wherein said first viral vector further comprises a CMV promoter to drive expression the Cas9 nuclease.

12. The delivery system according to claim 1, wherein said second viral vector does not include a nucleotide sequence encoding a reporter gene.

13. A self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said first and second viral vectors are not AAV8 viral vectors.

14. The delivery system according to claim 13, wherein said first and second viral vectors are selected from AAV1, AA2, AAV3, AAV4, AAV5, AAV6, AAV7, AAV9, AAV PHP.B and AAV PHP.eB.

15. (canceled)

16. (canceled)

17. (canceled)

18. (canceled)

19. A self-inactivating CRISPR/Cas9 delivery system comprising:
a first viral vector having an expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said first viral vector further comprises a CMV promoter to drive expression the Cas9 nuclease.

20. (canceled)

21. A self-inactivating CRISPR/Cas9 delivery system comprising:
a first viral vector having expression unit for expression of a Cas9 nuclease and a nucleotide sequence encoding an sgRNA targeting a specific genomic locus; and
a second viral vector having a nucleotide sequence encoding an sgRNA targeting expression of the Cas9 nuclease by the expression unit,
wherein said second viral vector does not include a nucleotide sequence encoding a reporter gene.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A pharmaceutical composition comprising a self-inactivating CRISPR/Cas9 delivery system according to claim 1 and a pharmaceutical acceptable carrier or excipient.

35. (canceled)

36. A method for treating a neurodegenerative disease related to genes in the CNS comprising administering to a patient in need thereof a self-inactivating CRISPR/Cas9 delivery system according to claim 1, wherein said sgRNA targets a specific genomic locus associated with said neurodegenerative disease.

37. The method according to claim 36, wherein said neurodegenerative disease is selected from Huntington's disease, Alzheimer's disease, Parkinson's disease, Amyotrophic Lateral Sclerosis (ALS), Frontotemporal dementia (FTD) (aka Pick's disease), spinocerebellar ataxi (SCA), particularly types 1 to 17, Retinitis Pigmentosa, and Age-related macular degeneration (AMD).

38. (canceled)

39. (canceled)

40. (canceled)

41. (canceled)

42. (canceled)

43. A method according to claim 6, wherein said system or composition is administered at a dosage of 10⁹ to 10¹⁴ viral genomes or infectious units of viral vectors per dose,

44. (canceled)


SEQUENCE LISTING (15 sequences)

SEQ ID NO: 1 - DNA, 21 bp, synthetic construct
  source 1..21: mol_type = other DNA
tggaaaagctgatgaaggcct

SEQ ID NO: 2 - DNA, 21 bp, synthetic construct
  source 1..21: mol_type = other DNA
gaaggccttcatcagcttttc

SEQ ID NO: 3 - DNA, 21 bp, synthetic construct
  source 1..21: mol_type = other DNA
ggtgatgccgatgtccaggcc

SEQ ID NO: 4 - DNA, 22 bp, synthetic construct
  source 1..22: mol_type = other DNA
gtgagccagaagctgagcgagg

SEQ ID NO: 5 - DNA, 22 bp, synthetic construct
  source 1..22: mol_type = other DNA
gaggtccctcatagtaggtccg

SEQ ID NO: 6 - DNA, 21 bp, synthetic construct
  source 1..21: mol_type = other DNA
gctcaggatgaagtcgtccac

SEQ ID NO: 7 - DNA, 21 bp, synthetic construct
  source 1..21: mol_type = other DNA
gttgttcagcagatcttccag

SEQ ID NO: 8 - DNA, 21 bp, synthetic construct
  source 1..21: mol_type = other DNA
gacttcatcaaccggaacctg

SEQ ID NO: 9 - DNA, 22 bp, synthetic construct
  source 1..22: mol_type = other DNA
gtgcccgagatcgaaaccgagc

SEQ ID NO: 10 - DNA, 22 bp, synthetic construct
  source 1..22: mol_type = other DNA
gatggaacagtacggcgacgag

SEQ ID NO: 11 - DNA, 22 bp, synthetic construct
  source 1..22: mol_type = other DNA
gacgccattgtccaggtacacg

SEQ ID NO: 12 - DNA, 23 bp, synthetic construct
  source 1..23: mol_type = other DNA
gaacatgatcgacatcacctacc

SEQ ID NO: 13 - DNA, 20 bp, synthetic construct
  source 1..20: mol_type = other DNA
gactatcatatgcttaccgt

SEQ ID NO: 14 - DNA, 20 bp, synthetic construct
  source 1..20: mol_type = other DNA
cgaaggctacgtccaggagc

SEQ ID NO: 15 - DNA, 21 bp, synthetic construct
  source 1..21: mol_type = other DNA
acaaccggtatggtgagcaag